(19) United States
(12) Patent Application Publication
(10) Pub. No.: US 2024/0335399 A1
(43) Pub. Date: Oct. 10, 2024

(54) PHENYLEPHRINE HYDROCHLORIDE-CONTAINING TABLET, PREPARATION METHOD AND USE

(71) Applicant: HQ PHARMA (SHANGHAI) CO., LTD., Shanghai (CN)

(72) Inventors: Dunwei He, Shanghai (CN); Jiezi Qin, Shanghai (CN); Yuzhang Hu, Shanghai (CN)

(73) Assignee: HQ PHARMA (SHANGHAI) CO., LTD., Shanghai (CN)

(21) Appl. No.: 18/271,188

(22) PCT Filed: Apr. 23, 2023
(86) PCT No.: PCT/CN2023/089967

§ 371 Date: Jul. 6, 2023

(30) Foreign Application Priority Data
Apr. 27, 2022 (CN) 202210454188.1

(51) Int. Cl. A61K 31/137; A61K 9/20; A61K 9/46; A61K 31/167; A61K 31/4402; A61K 31/485

(52) U.S. Cl. CPC A61K 31/137; A61K 9/0007; A61K 9/2009; A61K 9/2013; A61K 9/2018; A61K 9/2027; A61K 9/2036; A61K 9/205; A61K 9/2095; A61K 31/167; A61K 31/4402; A61K 31/485

(57) ABSTRACT

Disclosed are a phenylephrine hydrochloride-containing tablet, a preparation method and use. The method for preparing a phenylephrine hydrochloride-containing tablet includes: mixing a granule A with other granules to be uniform to obtain a mixture, and subjecting the mixture to tablet pressing to obtain the phenylephrine hydrochloride-containing tablet; wherein the granule A is prepared by dry granulation, and contains phenylephrine hydrochloride as a single active pharmaceutically ingredient, the other granules comprise at least one selected from the group consisting of a granule B and a granule C, and the granule B contains acetaminophen as an active pharmaceutical ingredient, and the granule C contains dextromethorphan hydrobromide and chlorpheniramine maleate as active pharmaceutical ingredients.

### CROSS REFERENCE TO RELATED APPLICATION

This patent application is a national stage application of International Patent Application No. PCT/CN2023/089967, filed on Apr. 23, 2023, which claims the priority of Chinese Patent Application No. CN2022104541881, entitled “Phenylephrine hydrochloride-containing tablet, preparation method and use” filed with the China National Intellectual Property Administration on Apr. 27, 2022, the disclosure of which is incorporated herein by reference in its entirety.

### TECHNICAL FIELD

The present disclosure belongs to the field of pharmaceutical technology, and in particular relates to a phenylephrine hydrochloride-containing tablet, a preparation method and use.

### BACKGROUND

Phenylephrine hydrochloride (C₉H₁₃NO₂.HCl, 203.67) is an adrenoceptor agonist, and is commonly used to prevent and treat hypotension caused by reasons such as spinal anesthesia, general anesthesia, and the administration of chlorpromazine and also used for supraventricular tachycardia and dilated mydriasis examination.

Since multiple active pharmaceutical ingredients (APIs) are commonly required to work together in the drug administration practice, the development of multi-API drugs is one of the important directions in current drug development. However, phenylephrine hydrochloride is significantly difficult to be applied in the development of multi-API drugs due to its own unstable nature and easy reaction with other active ingredients. For example, Gong Ya et al. reports that in solid formulations, phenylephrine hydrochloride can react with chlorpheniramine maleate to form an adduct, which limits the application of phenylephrine hydrochloride in multi-API drug development. Therefore, there is an urgent need to provide a feasible multi-API composition containing phenylephrine hydrochloride and a solution to improve the stability of phenylephrine hydrochloride in multi-API drugs.

### SUMMARY

An object of the present disclosure is to provide a method for preparing a multiple active pharmaceutical ingredients-containing tablet, and the active pharmaceutical ingredients comprise phenylephrine hydrochloride. In this method, the stability of phenylephrine hydrochloride is improved by the reasonable compounding of different active pharmaceutical ingredients.

Another object of the present disclosure is to provide a multiple active pharmaceutical ingredients drug prepared by the above method and use thereof, to solve the technical problem of instability of phenylephrine hydrochloride in the use of the existing phenylephrine hydrochloride-containing drugs.

To solve the above technical problems and achieve the above objects, the present disclosure provides the following technical solutions:

In the first aspect, the present disclosure provides a method for preparing a phenylephrine hydrochloride-containing tablet, comprising:
mixing a granule A with other granules to be uniform to obtain a mixture, and
subjecting the mixture to tablet pressing to obtain the phenylephrine hydrochloride-containing tablet;
wherein the granule A is prepared by dry granulation, and contains phenylephrine hydrochloride as a single active pharmaceutically ingredient,
the other granules comprise at least one selected from the group consisting of a granule B and a granule C, and
the granule B contains acetaminophen as an active pharmaceutical ingredient, and the granule C contains dextromethorphan hydrobromide and chlorpheniramine maleate as active pharmaceutical ingredients.

In some embodiments, a mass ratio of the active pharmaceutical ingredient contained in the granule B to phenylephrine hydrochloride is in a range of 20-80:1, and a mass ratio of the active pharmaceutical ingredients contained in the granule C to phenylephrine hydrochloride is in a range of 1.2-4.8:1.

In some embodiments, the mass ratio of the active pharmaceutical ingredient contained in the granule B to phenylephrine hydrochloride is in a range of 40-60:1, preferably 50:1, and the mass ratio of the active pharmaceutical ingredient contained in the granule C to phenylephrine hydrochloride is in a range of 2-2.8:1, preferably 2.4:1.

In some embodiments, the method further comprises adding an excipient during the preparation of the phenylephrine hydrochloride-containing tablet, and the excipient comprises calcium silicate and other common excipients.

In some embodiments, the other common excipients comprise at least one selected from the group consisting of:
a stabilizer, preferably calcium silicate;
a filler, preferably mannitol or maltodextrin;
a lubricant, preferably magnesium stearate;
a defoamer, preferably dimethicone;
a flavoring agent, preferably strawberry powder essence;
a pigment, preferably allure red (2-naphthalenesulfonicacid,6-hydroxy-5-[(2-methoxy-5-methyl-4-sulfophenyl)azo]);
an acid source, preferably anhydrous citric acid;
a sweetener, preferably sucralose;
a binder, preferably povidone K30; and
an alkali source, preferably potassium bicarbonate or sodium bicarbonate.

In some embodiments, during the preparation of the granule A, a mass ratio of the excipient to phenylephrine hydrochloride is in a range of 5-200:1, and a mass ratio of calcium silicate to phenylephrine hydrochloride is in a range of 1-10:1.

In some embodiments, the granule B and granule C are separately granulated, preferably the granule B and granule C are separately granulated by wet granulation.

In the second aspect, the present disclosure provides use of the method described in any one of the above embodiments in preparing a symptomatic treatment drug for colds.

In the third aspect, the present disclosure provides a tablet prepared by the method described in any one of the above embodiments.

In some embodiments, a dosage form of the tablet comprises one selected from the group consisting of an internal tablet, an oral tablet, a sublingual tablet, a topical tablet, a microcapsule tablet, an effervescent tablet, and a multilayer tablet.

In the present disclosure, the pharmaceutical composition containing multiple active ingredients could ensure the stability of phenylephrine hydrochloride by reasonable compounding of different API ingredients with phenylephrine hydrochloride. Meanwhile, the present disclosure provides a multi-API effervescent tablet drug prepared by using the above pharmaceutical composition and a preparation method thereof. In this method, on the basis of each ingredient provided by the above composition, phenylephrine hydrochloride is separately granulated by dry granulation, such that phenylephrine hydrochloride has good stability during the preparation and use of the prepared multi-API drug.

### DETAILED DESCRIPTION OF THE EMBODIMENTS

In order to make the objects, technical solutions, and advantages of the embodiments of the present disclosure clearer, the technical solutions in the embodiments of the present disclosure will be clearly and completely described below with reference to the embodiments of the present disclosure. It is clear that the embodiments described are a part of the embodiments of the present disclosure but not all of them. Therefore, based on the embodiments in the present disclosure, all other embodiments obtained by those skilled in the art without making creative labor shall fall within the protection scope of the present disclosure.

In specific embodiments, in the first aspect, the present disclosure provides a method for preparing a phenylephrine hydrochloride-containing tablet, comprising:
mixing a granule A with other granules to be uniform to obtain a mixture, and
subjecting the mixture to tablet pressing to obtain the phenylephrine hydrochloride-containing tablet;
wherein the granule A is prepared by dry granulation, and contains phenylephrine hydrochloride as a single active pharmaceutically ingredient,
the other granules comprise at least one selected from the group consisting of a granule B and a granule C; and
the granule B contains acetaminophen as an active pharmaceutical ingredient, and the granule C contains dextromethorphan hydrobromide and chlorpheniramine maleate as active pharmaceutical ingredients.

In optional embodiments, a mass ratio of the active pharmaceutical ingredient contained in the granule B to phenylephrine hydrochloride is in a range of 20-80:1, and a mass ratio of the active pharmaceutical ingredient contained in the granule C to phenylephrine hydrochloride is in a range of 1.2-4.8:1.

In optional embodiments, the mass ratio of the active pharmaceutical ingredient contained in the granule B to phenylephrine hydrochloride is in a range of 40-60:1, preferably 50:1, and the mass ratio of the active pharmaceutical ingredient contained in the granule C to phenylephrine hydrochloride is in a range of 2-2.8:1, preferably 2.4:1.

In optional embodiments, the method further comprises adding an excipient during the preparation of the phenylephrine hydrochloride-containing tablet, and the excipient comprises calcium silicate and other common excipients.

Calcium silicate is a porous material and could encapsulate phenylephrine hydrochloride in micro-pores, which could avoid the effect of light and oxygen on phenylephrine hydrochloride, and also reduce the contact of other active ingredients with phenylephrine hydrochloride, thus improving the stability of phenylephrine hydrochloride. It should be understood that the other common excipients refer to those excipient components that do not affect the stability of phenylephrine hydrochloride and could be conventionally selected by those skilled in the art among the available excipients. At the same time, the addition of the excipient component will not change the function relationship between the above multi-API.

In optional embodiments, the other common excipients comprise at least one selected from the group consisting of:
a stabilizer, preferably calcium silicate;
a filler, preferably mannitol or maltodextrin;
a lubricant, preferably magnesium stearate;
a defoamer, preferably dimethicone;
a flavoring agent, preferably strawberry powder essence;
a pigment, preferably allure red;
an acid source, preferably anhydrous citric acid;
a sweetener, preferably sucralose;
a binder, preferably povidone K30; and
an alkali source, preferably potassium bicarbonate or sodium bicarbonate.

In optional embodiments, during the preparation of the granule A, a mass ratio of the excipient to phenylephrine hydrochloride is in a range of 5-200:1, and a mass ratio of calcium silicate to phenylephrine hydrochloride is in a range of 1-10:1.

In optional embodiments, the granule B and granule C are separately granulated, preferably the granule B and granule C are separately granulated by wet granulation.

In the second aspect, the present disclosure provides use of the method of any one of the above embodiments in preparing a symptomatic treatment drug for colds.

In the third aspect, the present disclosure provides a tablet prepared by the method of any one of the above embodiments.

In optional embodiments, a dosage form of the tablet comprises one selected from the group consisting of an internal tablet, an oral tablet, a sublingual tablet, a topical tablet, a microcapsule tablet, an effervescent tablet, and a multilayer tablet.

It is understood that for the specific tablet dosage forms described in the preceding embodiments, those skilled in the art could adjust the tablet dosage form by conventional means with reference to conventional molding preparation methods. Therefore, in the case where other dosage forms have been limited in the preceding embodiment, it should be understood that the preparation methods of other solid dosage forms that have not been substantially changed compared to the above methods for preparing each tablet dosage form shall also fall within the protection scope of the present disclosure. The method for preparing an effervescent tablet described in the embodiments of the present disclosure is only one of the examples of the method for preparing solid dosage forms.

Some embodiments of the present disclosure are described in detail below. The following examples and the features in the examples may be combined with each other without conflict.

### EXAMPLE 1

This example provided a prescription of a pharmaceutical composition that was shown as follows, and a method for preparing an effervescent tablet by using the prescription:

| Name | | mg/tablet |
|---|---|---|
| Granule A | Phenylephrine hydrochloride | 5 |
| | Mannitol | 200 |
| | Maltodextrin | 200 |
| | Calcium silicate | 20 |
| | Magnesium stearate | 5 |
| Granule B | Paracetamol | 250 |
| | Anhydrous citric acid | 900 |
| | Mannitol | 200 |
| | Sucralose | 10 |
| | Povidone K30 | 20 |
| | Allure red | 2 |
| Granule C | Dextromethorphan | 10 |
| | hydrobromide | |
| | Chlorpheniramine maleate | 2 |
| | Sodium bicarbonate | 205 |
| | Potassium bicarbonate | 1300 |
| | Povidone K30 | 20 |
| Additional | Magnesium stearate | 20 |
| excipient | Calcium silicate | 40 |
| | Dimethicone | 20 |
| | Strawberry powder essence | 40 |

The method was performed by the following procedures:
1. Preparation of granule A: Raw materials and excipients were weighed. Phenylephrine hydrochloride was mixed with calcium silicate, mixed with maltodextrin in equal increments, and mixed with mannitol and magnesium stearate successively, obtaining a first mixture. The first mixture was pressed into flakes and granulated with a dry granulator, obtaining the granule A.
2. Preparation of granule B: Raw materials and excipients were weighed. Allure red was dissolved in water, obtaining an allure red aqueous solution. The rest of the raw materials and excipients were mixed in a wet granulator, and the allure red aqueous solution was added thereto, obtaining a first soft material. The first soft material was granulated with a swing granulator, obtaining a first granule. The first granule was dried with a fluid bed and subjected to milling granule with the swing granulator, obtaining the granule B
3. Preparation of granule C: Raw materials and excipients were weighed. The excipients were mixed with the raw materials in equal increments in the wet granulator, and water was added thereto, obtaining a second soft material. The second soft material was granulated with the swing granulator, obtaining second granules. The second granules were dried with the fluid bed and subjected to milling granule with the swing granulator, obtaining the granule C.
4. Mixing: The granules and additional excipients were weighed. Calcium silicate adsorbed dimethicone and then was mixed with the granules and strawberry powder essence, and then magnesium stearate lubrication was added thereto, obtaining a second mixture.
5. The second mixture was pressed into a 24 mm-round stamping tablet with a rotary press, obtaining an effervescent tablet. The ambient humidity was controlled at a relative humidity of 40% or less.

### EXAMPLE 2

This example provided a prescription of a pharmaceutical composition that was shown as follows, and a method for preparing an effervescent tablet by using the prescription:

| Name | | mg/tablet |
|---|---|---|
| Granule A | Phenylephrine hydrochloride | 5 |
| | Mannitol | 200 |
| | Maltodextrin | 200 |
| | Calcium silicate | 20 |
| | Magnesium stearate | 5 |
| Granule B | Paracetamol | 100 |
| | Anhydrous citric acid | 900 |
| | Mannitol | 200 |
| | Sucralose | 10 |
| | Povidone K30 | 20 |
| | Allure red | 2 |
| Granule C | Dextromethorphan | 5 |
| | hydrobromide | |
| | Chlorpheniramine maleate | 1 |
| | Sodium bicarbonate | 205 |
| | Potassium bicarbonate | 1300 |
| | Povidone K30 | 20 |
| Additional | Magnesium stearate | 20 |
| excipient | Calcium silicate | 40 |
| | Dimethicone | 20 |
| | Strawberry powder essence | 40 |

The preparation method was the same as Example 1.

### EXAMPLE 3

This example provided a prescription of a pharmaceutical composition that was shown as follows, and a method for preparing an effervescent tablet by using the prescription:

| Name | | mg/tablet |
|---|---|---|
| Granule A | Phenylephrine hydrochloride | 5 |
| | Mannitol | 200 |
| | Maltodextrin | 200 |
| | Calcium silicate | 20 |
| | Magnesium stearate | 5 |
| Granule B | Paracetamol | 400 |
| | Anhydrous citric acid | 900 |
| | Mannitol | 200 |
| | Sucralose | 10 |
| | Povidone K30 | 20 |
| | Allure red | 2 |
| Granule C | Dextromethorphan | 20 |
| | hydrobromide | |
| | Chlorpheniramine maleate | 4 |
| | Sodium bicarbonate | 205 |
| | Potassium bicarbonate | 1300 |
| | Povidone K30 | 20 |
| Additional | Magnesium stearate | 20 |
| excipient | Calcium silicate | 40 |
| | Dimethicone | 20 |
| | Strawberry powder essence | 40 |

The preparation method was the same as Example 1.

### EXAMPLE 4

This example provided a prescription of a pharmaceutical composition that was shown as follows, and a method for preparing an effervescent tablet by using the prescription:

| Name | | mg/tablet |
|---|---|---|
| Granule A | Phenylephrine hydrochloride | 5 |
| | Mannitol | 10 |
| | Maltodextrin | 9.5 |
| | Calcium silicate | 5 |
| | Magnesium stearate | 0.5 |
| Granule B | Paracetamol | 250 |
| | Anhydrous citric acid | 900 |
| | Mannitol | 200 |
| | Sucralose | 10 |
| | Povidone K30 | 20 |
| | Allure red | 2 |
| Granule C | Dextromethorphan | 10 |
| | hydrobromide | |
| | Chlorpheniramine maleate | 2 |
| | Sodium bicarbonate | 205 |
| | Potassium bicarbonate | 1300 |
| | Povidone K30 | 20 |
| Additional | Magnesium stearate | 20 |
| excipient | Calcium silicate | 40 |
| | Dimethicone | 20 |
| | Strawberry powder essence | 40 |

The preparation method was the same as Example 1.

### EXAMPLE 5

This example provided a prescription of a pharmaceutical composition that was shown as follows, and a method for preparing an effervescent tablet by using the prescription:

| Name | | mg/tablet |
|---|---|---|
| Granule A | Phenylephrine hydrochloride | 5 |
| | Mannitol | 500 |
| | Maltodextrin | 440 |
| | Calcium silicate | 50 |
| | Magnesium stearate | 10 |
| Granule B | Paracetamol | 250 |
| | Anhydrous citric acid | 900 |
| | Mannitol | 200 |
| | Sucralose | 10 |
| | Povidone K30 | 20 |
| | Allure red | 2 |
| Granule C | Dextromethorphan | 10 |
| | hydrobromide | |
| | Chlorpheniramine maleate | 2 |
| | Sodium bicarbonate | 205 |
| | Potassium bicarbonate | 1300 |
| | Povidone K30 | 20 |
| Additional | Magnesium stearate | 20 |
| excipient | Calcium silicate | 40 |
| | Dimethicone | 20 |
| | Strawberry powder essence | 40 |

The preparation method was the same as Example 1.

### COMPARATIVE EXAMPLE 1

This example provided a prescription of a pharmaceutical composition that was shown as follows, and a method for preparing an effervescent tablet by using the prescription:

| Name | | mg/tablet |
|---|---|---|
| Granule I | Paracetamol | 250 |
| | Phenylephrine hydrochloride | 5 |
| | Anhydrous citric acid | 900 |
| | Mannitol | 400 |
| | Maltodextrin | 200 |
| | Sucralose | 10 |
| | Povidone K30 | 20 |
| | Allure red | 2 |
| Granule II | Dextromethorphan | 10 |
| | hydrobromide | |
| | Chlorpheniramine maleate | 2 |
| | Sodium bicarbonate | 205 |
| | Potassium bicarbonate | 1300 |
| | Povidone K30 | 20 |
| Additional | Magnesium stearate | 25 |
| excipient | Calcium silicate | 60 |
| | Dimethicone | 20 |
| | Strawberry powder essence | 40 |

Preparation method was performed by the following procedures:
1. Preparation of granule I: Raw materials and excipients were weighed. Allure red was dissolved in water, obtaining an allure red aqueous solution. Phenylephrine hydrochloride was mixed with maltodextrin in equal increments, mixed with mannitol and the rest of the raw materials and excipients successively in a wet granulator, and the allure red aqueous solution was added thereto, obtaining a first soft material. The first soft material was granulated with a swing granulator, obtaining a first granule. The first granule was dried with a fluid bed and subjected to milling granule with the swing granulator, obtaining the granule I.
2. Preparation of granule II: Raw materials and excipients were weighed. The excipients were mixed with the raw materials in equal increments in the wet granulator, and water was added thereto, obtaining a second soft material. The second soft material was granulated with the swing granulator, obtaining a second granule. The second granule was dried with the fluid bed and subjected to milling granule with the swing granulator, obtaining the granule II.
3. Mixing: The granules and additional excipients were weighed. Calcium silicate adsorbed dimethicone and then was mixed with the granules and strawberry powder essence, and then magnesium stearate lubrication was added thereto, obtaining a mixture.
4. The mixture was pressed into a 24 mm-round stamping tablet with a rotary press, obtaining an effervescent tablet. The ambient humidity was controlled at a relative humidity of 40% or less.

### COMPARATIVE EXAMPLE 2

This example provided a prescription of a pharmaceutical composition that was shown as follows, and a method for preparing an effervescent tablet by using the prescription:

The prescription was as follows:

| Name | | mg/tablet |
|---|---|---|
| Granule I | Paracetamol | 250 |
| | Anhydrous citric acid | 900 |
| | Mannitol | 200 |
| | Sucralose | 10 |
| | Povidone K30 | 20 |
| | Allure red | 2 |
| Granule II | Dextromethorphan | 10 |
| | hydrobromide | |
| | Chlorpheniramine maleate | 2 |
| | Phenylephrine hydrochloride | 5 |
| | Mannitol | 200 |
| | Maltodextrin | 200 |
| | Sodium bicarbonate | 205 |
| | Potassium bicarbonate | 1300 |
| | Povidone K30 | 20 |
| Additional | Magnesium stearate | 25 |
| excipient | Calcium silicate | 60 |
| | Dimethicone | 20 |
| | Strawberry powder essence | 40 |

Preparation method was performed by the following procedures:
1. Preparation of granule I: Raw excipients were weighed. Allure red was dissolved in water, obtaining an allure red aqueous solution. The rest of the raw materials and excipients were mixed in a wet granulator, and the allure red aqueous solution was added thereto, obtaining a first soft material. The first soft material was granulated with a swing granulator, obtaining a first granule. The first granule was dried with a fluid bed and subjected to milling granule with the swing granulator, obtaining the granule I.
2. Preparation of granule II: Raw materials and excipients were weighed. Raw materials were mixed with maltodextrin in equal increments, and mixed with mannitol and the rest of the raw materials and excipients successively in the wet granulator, and water was added thereto, obtaining a second soft material. The second soft material was granulated with the swing granulator, obtaining a second granule. The second granule was dried with the fluid bed and subjected to milling granule with the swing granulator, obtaining the granule II.
3. Mixing: The granules and additional excipients were weighed. Calcium silicate adsorbed dimethicone and then was mixed with the granules and strawberry powder essence, and then magnesium stearate was added thereto for lubrication, obtaining a mixture.
4. The mixture was pressed into a 24 mm-round stamping tablet with a rotary press, obtaining an effervescent tablet. The ambient humidity was controlled at a relative humidity of 40% or less.

### COMPARATIVE EXAMPLE 3

This example provided a prescription of a pharmaceutical composition that was shown as follows, and a method for preparing an effervescent tablet by using the prescription:

The prescription was as follows:

| Name | | mg/tablet |
|---|---|---|
| Granule I | Paracetamol | 250 |
| | Anhydrous citric acid | 900 |
| | Mannitol | 200 |
| | Sucralose | 10 |
| | Povidone K30 | 20 |
| | Allure red | 2 |
| Granule II | Dextromethorphan | 10 |
| | hydrobromide | |
| | Chlorpheniramine maleate | 2 |
| | Sodium bicarbonate | 205 |
| | Potassium bicarbonate | 1300 |
| | Povidone K30 | 20 |
| Granule III | Phenylephrine hydrochloride | 5 |
| | Mannitol | 200 |
| | Maltodextrin | 200 |
| | Magnesium stearate | 5 |
| Additional | Magnesium stearate | 20 |
| excipient | Calcium silicate | 60 |
| | Dimethicone | 20 |
| | Strawberry powder essence | 40 |

Preparation method was performed by the following procedures:
1. Preparation of granule I: Raw materials and excipients were weighed. Allure red was dissolved in water, obtaining an allure red aqueous solution. The rest of the raw materials and excipients were mixed in a wet granulator, and the allure red aqueous solution was added thereto, obtaining a first soft material. The first soft material was granulated with a swing granulator, obtaining a first granule. The first granule was dried with a fluid bed and subjected to milling granule with the swing granulator, obtaining the granule I.
2. Preparation of granule II: Raw materials and excipients were weighed. The excipients were mixed with the raw materials in equal increments in the wet granulator, and water was added thereto, obtaining a second soft material. The second soft material was granulated with a swing granulator, obtaining a second granule. The second granule was dried with a fluid bed and subjected to milling granule with the swing granulator, obtaining the granule II.
3. Preparation of granule III: Raw materials and excipients were weighed. Phenylephrine hydrochloride was mixed with maltodextrin in equal increments, and mixed with mannitol and magnesium stearate successively, obtaining a first mixture. The first mixture was pressed into flakes and granulated with a dry granulator, obtaining the granule III.
4. Mixing: The granules and additional excipients were weighed. Calcium silicate adsorbed dimethicone and then was mixed with the granules and strawberry powder essence, and then magnesium stearate was added thereto for lubrication, obtaining a second mixture.
5. The second mixture was pressed into a 24 mm-round stamping tablet with a rotary press, obtaining an effervescent tablet. The ambient humidity was controlled at a relative humidity of 40% or less.

### COMPARATIVE EXAMPLE 4

This example provided a prescription of a pharmaceutical composition that was shown as follows, and a method for preparing an effervescent tablet by using the prescription:

The prescription was as follows:

| Name | | mg/tablet |
|---|---|---|
| Granule I | Paracetamol | 250 |
| | Anhydrous citric | 900 |
| | acid | |
| | Mannitol | 200 |
| | Sucralose | 10 |
| | Povidone K30 | 20 |
| | Allure red | 2 |
| Granule II | Chlorpheniramine | 2 |
| | maleate | |
| | Sodium | 205 |
| | bicarbonate | |
| | Potassium | 1300 |
| | bicarbonate | |
| | Povidone K30 | 20 |
| Granule III | Phenylephrine | 5 |
| | hydrochloride | |
| | Dextromethorphan | 10 |
| | hydrobromide | |
| | Calcium silicate | 20 |
| | Mannitol | 200 |
| | Maltodextrin | 200 |
| | Magnesium | 5 |
| | stearate | |
| Additional | Magnesium | 20 |
| excipient | stearate | |
| | Calcium silicate | 40 |
| | Dimethicone | 20 |
| | Strawberry | 40 |
| | powder essence | |

Preparation method was performed by the following procedures:
1. Preparation of granule I: Raw materials and excipients were weighed. Allure red was dissolved in water, obtaining an allure red aqueous solution. The rest of the raw materials and excipients were mixed in a wet granulator, and the allure red aqueous solution was added thereto, obtaining a first soft material. The first soft material was granulated with a swing granulator, obtaining a first granule. The first granule was dried with a fluid bed and subjected to milling granule with the swing granulator, obtaining the granule I.
2. Preparation of granule II: Raw materials and excipients were weighed. The excipients were mixed with the raw materials in equal increments in the wet granulator, and water was added thereto, obtaining a second soft material. The second soft material was granulated with a swing granulator, obtaining a second granule. The second granule was dried with a fluid bed and subjected to milling granule with the swing granulator, obtaining the granule II.
3. Preparation of granule III: Raw materials and excipients were weighed. Phenylephrine hydrochloride was mixed with dextromethorphan hydrobromide and calcium silicate, then mixed with maltodextrin in equal increments, and mixed with mannitol and magnesium stearate successively, obtaining a first mixture. The first mixture was pressed into flakes and granulated with a dry granulator, obtaining the granule III.
4. Mixing: The granules and additional excipients were weighed. Calcium silicate adsorbed dimethicone and then was mixed with the granules and strawberry powder essence, and then magnesium stearate was added thereto for lubrication, obtaining a second mixture.
5. The second mixture was pressed into a 24 mm-round stamping tablet with a rotary press, obtaining an effervescent tablet. The ambient humidity was controlled at a relative humidity of 40% or less.

### COMPARATIVE EXAMPLE 5

This example provided a prescription of a pharmaceutical composition that was shown as follows, and a method for preparing an effervescent tablet by using the prescription:

The prescription was as follows:

| Name | | mg/tablet |
|---|---|---|
| Granule I | Paracetamol | 250 |
| | Anhydrous citric | 900 |
| | acid | |
| | Mannitol | 200 |
| | Sucralose | 10 |
| | Povidone K30 | 20 |
| | Allure red | 2 |
| Granule II | Dextromethorphan | 10 |
| | hydrobromide | |
| | Sodium | 205 |
| | bicarbonate | |
| | Potassium | 1300 |
| | bicarbonate | |
| | Povidone K30 | 20 |
| Granule III | Phenylephrine | 5 |
| | hydrochloride | |
| | Chlorpheniramine | 2 |
| | maleate | |
| | Calcium silicate | 20 |
| | Mannitol | 200 |
| | Maltodextrin | 200 |
| | Magnesium | 5 |
| | stearate | |
| Additional | Magnesium | 20 |
| excipient | stearate | |
| | Calcium silicate | 40 |
| | Dimethicone | 20 |
| | Strawberry powder | 40 |
| | essence | |

Preparation method was performed by the following procedures:
1. Preparation of granule I: Raw materials and excipients were weighed. Allure red was dissolved in water, obtaining an allure red aqueous solution. The rest raw materials and excipients were mixed in a wet granulator, and the allure red aqueous solution was added thereto, obtaining a first soft material. The first soft material was granulated with a swing granulator, obtaining a first granule. The first granule was dried with a fluid bed and subjected to milling granule with the swing granulator, obtaining the granule I.
2. Preparation of granule II: Raw materials and excipients were weighed. The excipients were mixed with raw materials in equal increments in the wet granulator, and water was added thereto, obtaining a second soft material. The second soft material was granulated with a swing granulator, obtaining a second granule. The second granule was dried with a fluid bed and subjected to milling granule with the swing granulator, obtaining the granule II.
3. Preparation of granule III: Raw materials and excipients were weighed. Phenylephrine hydrochloride was mixed with chlorpheniramine maleate and calcium silicate, then mixed with maltodextrin in equal increments, and mixed with mannitol and magnesium stearate successively, obtaining a first mixture. The first mixture was pressed into flakes and granulated with a dry granulator, obtaining the granule III.
4. Mixing: The granules and additional excipients were weighed. Calcium silicate adsorbed dimethicone and then was mixed with the granules and strawberry powder essence, and then magnesium stearate lubrication was added thereto, obtaining a second mixture.
5. The second mixture was pressed into a 24 mm-round stamping tablet with a rotary press, obtaining an effervescent tablet. The ambient humidity was controlled at a relative humidity of 40% or less.

### EXPERIMENTAL EXAMPLE 1

1. Phenylephrine hydrochloride in the effervescent tablets prepared in Examples 1 to 6 was assayed by using high-performance liquid chromatography according to the following chromatographic conditions:

| Instrument | High-performance liquid chromatograph | | |
|---|---|---|---|
| Chromatographic | GL Inertsil ODS-3V 4.6 mm × 150 mm, 5 μm | | |
| column | | | |
| Moving phase | Moving phase A: 0.02 mol/L ammonium acetate (pH value was | | |
| | adjusted to 4.5 with glacial acetic acid):methanol = 9:1 | | |
| | Moving phase B: Methanol | | |
| | Time min | Moving phase A % | Moving phase B % |
| | 0 | 100 | 0 |
| | 5 | 100 | 0 |
| | 15 | 70 | 30 |
| | 25 | 40 | 60 |
| | 26 | 100 | 0 |
| | 35 | 100 | 0 |
| Detecting | 280 nm | | |
| wavelength | | | |

| | |
|---|---|
| Column temperature | 30° C. |
| Flow rate | 1.0 mL/min |
| Injection volume | 10 μL |
| Diluent | 10% of Methanol |
| Control solution | Acetaminophen, 100 mg, 10 mg, 20 mg, and 10 mg of |
| | chlorpheniramine maleate, dextromethorphan hydrobromide, and |
| | phenylephrine hydrochloride were respectively weighed precisely, and |
| | placed into 10 mL, 25 mL, 10 mL, and 10 mL measuring flasks, |
| | respectively. An appropriate amount of diluent was added into each |
| | measuring flask for ultrasonic dissolution. The obtained dissolved |
| | solutions were stood to room temperature, and then dissolved with a |
| | diluent to a constant volume as a control stock solution. 5 mL, 1 mL, 1 |
| | mL, and 1 mL of each stock solution were pipetted precisely into a 20 |
| | mL flask and diluted to a scale with a diluent. |
| Test solution | 10 tablets were placed into 250 mL-conical flasks, respectively. 100 |
| | mL of a diluent was added to each conical flask and shaken at a speed |
| | of about 250 rpm for half an hour to obtain a diluted solution. Each |
| | diluted solution was filtered through a 0.45 μm-PTFE filter membrane. |
| Measurement and | Calculation by the external standard method, using the average value |
| calculation | of 10 tablets as the content value. |

2. Phenylephrine hydrochloride-related substances of the effervescent tablets prepared in Example 1 and Comparative Examples 1 to 5 were assayed by using high-performance liquid chromatography according to the following chromatographic conditions. The relevant substances are:
Phenylephrine hydrochloride impurity C, C9H11NO2.HCl, 201.65, 1-(3-Hydroxyphenyl)-2-(methylamino)ethan-1-one hydrochloride, which has a molecular structure formula shown as follows:

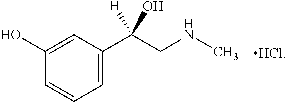

Phenylephrine hydrochloride impurity I, C10H13NO2, 179.22, 2-Methyl-1,2,3,4-tetrahydroisoquinoline-4,6-diol, which has a molecular structure formula shown as follows:

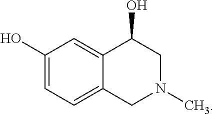 [SMILES: CN1Cc2ccc(O)cc2[C@@H](O)C1]

Phenylephrine hydrochloride impurity II, C7H6O2, 122.12, 3-Hydroxybenzaldehyde, which has a molecular structure formula shown as follows:

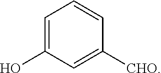

Chromatographic detection conditions:

| Instrument | High-performance liquid chromatograph | | |
|---|---|---|---|
| Chromatographic | Agilent Zorbax Rx C8 4.6 mm × 250 mm, 5 μm | | |
| column | | | |
| Mobile phase | Moving phase A: acetonitrile-pH 3.0 buffered solution 1 (which was | | |
| | prepared by weighing 1 g of sodium octanesulfonate monohydrate, | | |
| | adding 1000 mL of water thereto to obtain a mixture, stirring the | | |
| | mixture for dissolution, and adjusting pH value to 3.0 with phosphoric | | |
| | acid) = 10:90 | | |
| | Moving phase B: acetonitrile-pH 3.0 buffered solution 2 (which was | | |
| | prepared by weighing 11.5 g of ammonium dihydrogen phosphate and | | |
| | 1 g of sodium octanesulfonate , adding 1000 ml of water thereto to | | |
| | obtain a mixture, stirring the mixture for dissolution, and adjusting pH | | |
| | value to 3.0 with phosphoric acid) = 60:40 | | |
| | Time min | Moving phase A % | Moving phase B % |
| | 0 | 100 | 0 |
| | 5 | 100 | 0 |
| | 60 | 70 | 52 |
| | 65 | 40 | 100 |
| | 75 | 100 | 100 |
| | 80 | 100 | 0 |
| | 90 | 100 | 0 |
| Detecting | 225 nm | | |
| wavelength | | | |

| | |
|---|---|
| Column temperature | 20° C. |
| Flow rate | 1.5 mL/min |
| Injection volume | 50 μL |
| Sample tray kongwen | 10° C. |
| Diluent | 10% of Methanol |
| Control solution | Appropriate amounts of phenylephrine hydrochloride, phenylephrine |
| | hydrochloride impurity C, phenylephrine hydrochloride impurity I, and |
| | phenylephrine hydrochloride impurity II were weighed precisely, |
| | respectively, and then diluted with 10% methanol to a concentration of |
| | 0.5 mg of phenylephrine hydrochloride per 1 mL, 1.5 μg of |
| | phenylephrine hydrochloride impurity C per 1 mL, 5 μg of |
| | phenylephrine hydrochloride impurity I per 1 mL, and 1.5 μg of |
| | phenylephrine hydrochloride impurity II per 1 mL. |
| Test solution | 5 tablets were placed into 100 mL-conical flasks, respectively. 50 mL |
| | of a diluent was added to each conical flask, left for 10 min, then |
| | transferred to an oscillator and shaken for 10 min to obtain a diluted |
| | solution. Each diluted solution was filtered through a 0.45 μm PTFE |
| | filter membrane. |
| Measurement and | Calculation by the external standard method |
| calculation | |

The results of the above tests for phenylephrine hydrochloride and related substances are as follows:

| | | | Related substances (%) | | |
|---|---|---|---|---|---|
| Stability | | Content | Impuri- | Impuri- | Impuri- |
| time | Prescription | (%) | ty C | ty I | ty II |
| 0 Day | Example 1 | 97.1 | <0.1% | <0.1% | <0.1% |
| | Comparative | 96.3 | 0.3 | <0.1% | <0.1% |
| | Example 1 | | | | |
| | Comparative | 96.8 | 0.2 | <0.1% | <0.1% |
| | Example 2 | | | | |
| | Comparative | 97.7 | <0.1% | <0.1% | <0.1% |
| | Example 3 | | | | |
| | Comparative | 99.2 | <0.1% | <0.1% | <0.1% |
| | Example 4 | | | | |
| | Comparative | 98.5 | <0.1% | <0.1% | <0.1% |
| | Example 5 | | | | |
| After 3 months | Example 1 | 96.7 | <0.1% | <0.1% | <0.1% |
| at 30° C. ± 2° | Comparative | 95.8 | 0.6 | <0.1% | <0.1% |
| C./65% RH ± | Example 1 | | | | |
| 5% RH | Comparative | 96.1 | 0.5 | <0.1% | <0.1% |
| | Example 2 | | | | |
| | Comparative | 97.2 | 0.1 | <0.1% | <0.1% |
| | Example 3 | | | | |
| | Comparative | 99.0 | 0.1 | <0.1% | <0.1% |
| | Example 4 | | | | |
| | Comparative | 98.5 | 0.2 | <0.1% | <0.1% |
| | Example 5 | | | | |
| After 6 months | Example 1 | 96.9 | 0.1 | <0.1% | <0.1% |
| at 30° C. ± 2° | Comparative | 95.1 | 0.8 | <0.1% | <0.1% |
| C./65% RH ± | Example 1 | | | | |
| 5% RH | Comparative | 94.3 | 0.7 | <0.1% | <0.1% |
| | Example 2 | | | | |
| | Comparative | 97.4 | 0.2 | <0.1% | <0.1% |
| | Example 3 | | | | |
| | Comparative | 98.4 | 0.2 | <0.1% | <0.1% |
| | Example 4 | | | | |
| | Comparative | 98.2 | 0.3 | <0.1% | <0.1% |
| | Example 5 | | | | |

From the above test results, it can be seen that the granules without other active ingredients prepared by mixing phenylephrine hydrochloride and calcium silicate and then subjecting to dry granulation have the best stability. In Example 1, phenylephrine hydrochloride was granulated by dry method, and in Comparative Example 1 and Comparative Example 2, phenylephrine hydrochloride was granulated together with other active ingredients by wet method. It can be seen that the contents of all impurities in the effervescent tablets prepared by dry granulation of phenylephrine hydrochloride alone are less than 0.1% after 3 months of storage; after 6 months of storage, the content of impurity C is only 0.1%, while the contents of the other two impurities are still less than 0.1%, showing excellent stability. Compared with Example 1, the same dry granulation is used in Comparative Example 3, but the granules do not contain the stabilizer calcium silicate. It can be seen that the content of impurity C contained in the tablets exceeds 0.1% after long-term storage. In Comparative Example 4, dextromethorphan hydrobromide and phenylephrine hydrochloride are granulated together by dry granulation, and in Comparative Example 5, chlorpheniramine maleate and phenylephrine hydrochloride are granulated together by dry granulation. After 3 and 6 months of storage, the contents of impurity C contained in the tablets prepared in Comparative Example 4-5 are significantly increased, demonstrating the importance of separate granulation.

Finally, it should be noted that the above embodiments are only used to illustrate the technical solutions of the present disclosure but not limitations. Although the present disclosure has been described in detail with reference to the preceding embodiments, it should be understood by those skilled in the art that they can still modify the technical solutions described in the preceding embodiments or make equivalent substitutes for some or all of the technical features. However, these modifications or substitutes do not make the essence of the corresponding technical solutions out of the scope of the technical solutions of the embodiments of the present disclosure.

What is claimed is:

1. A method for preparing a phenylephrine hydrochloride-containing tablet, comprising:
mixing a granule A with other granules to be uniform to obtain a mixture, and
subjecting the mixture to tablet pressing to obtain the phenylephrine hydrochloride-containing tablet;
wherein the granule A is prepared by dry granulation, and contains phenylephrine hydrochloride as a single active pharmaceutically ingredient,
the other granules comprise at least one selected from the group consisting of a granule B and a granule C, and
the granule B contains acetaminophen as an active pharmaceutical ingredient, and the granule C contains dextromethorphan hydrobromide and chlorpheniramine maleate as active pharmaceutical ingredients.

2. The method of claim 1, wherein a mass ratio of the active pharmaceutical ingredient contained in the granule B to phenylephrine hydrochloride is in a range of 20-80:1, and a mass ratio of the active pharmaceutical ingredients contained in the granule C to phenylephrine hydrochloride is in a range of 1.2-4.8:1.

3. The method of claim 2, wherein the mass ratio of the active pharmaceutical ingredient contained in the granule B to phenylephrine hydrochloride is in a range of 40-60:1, and the mass ratio of the active pharmaceutical ingredients contained in the granule C to phenylephrine hydrochloride is in a range of 2-2.8:1.

4. The method of claim 3, wherein the mass ratio of the active pharmaceutical ingredient contained in the granule B to phenylephrine hydrochloride is 50:1, and the mass ratio of the active pharmaceutical ingredients contained in the granule C to phenylephrine hydrochloride is 2.4:1.

5. The method of claim 1, further comprising adding an excipient during the preparation of the phenylephrine hydrochloride-containing tablet, and the excipient comprises calcium silicate and other common excipients.

6. The method of claim 5, wherein the other common excipients comprise at least one selected from the group consisting of:
a stabilizer;
a filler;
a lubricant;
a defoamer;
a flavoring agent;
a pigment;
an acid source;
a sweetener;
a binder; and
an alkali source.

7. The method of claim 6, wherein the stabilizer is calcium silicate;
the filler is one selected from the group consisting of mannitol and maltodextrin;
the lubricant is magnesium stearate;
the defoamer is dimethicone;
the flavoring agent is strawberry powder essence;
the pigment is allure red;
the acid source is anhydrous citric acid;
the sweetener is sucralose;
the binder is povidone K30; and
the alkali source is one selected from the group consisting of potassium bicarbonate and sodium bicarbonate.

8. The method of claim 5, wherein during the preparation of the granule A, a mass ratio of the excipient to phenylephrine hydrochloride is in a range of 5-200:1, and a mass ratio of calcium silicate to phenylephrine hydrochloride is in a range of 1-10:1.

9. The method of claim 5, wherein an ambient humidity of the dry granulation is controlled at a relative humidity of not large than 40%.

10. The method of claim 5, wherein the granule B and granule C are separately granulated.

11. The method of claim 10, wherein the granule B and granule C are separately granulated by wet granulation.

12. A phenylephrine hydrochloride-containing tablet prepared by the method of claim 1.

13. The phenylephrine hydrochloride-containing tablet of claim 12, wherein a dosage form of the tablet comprises one selected from the group consisting of an internal tablet, an oral tablet, a sublingual tablet, a topical tablet, a microcapsule tablet, an effervescent tablet, and a multilayer tablet.

14. The method of claim 2, further comprising adding an excipient during the preparation of the phenylephrine hydrochloride-containing tablet, and the excipient comprises calcium silicate and other common excipients.

15. The method of claim 3, further comprising adding an excipient during the preparation of the phenylephrine hydrochloride-containing tablet, and the excipient comprises calcium silicate and other common excipients.

16. The method of claim 4, further comprising adding an excipient during the preparation of the phenylephrine hydrochloride-containing tablet, and the excipient comprises calcium silicate and other common excipients.

17. The phenylephrine hydrochloride-containing tablet of claim 13, wherein a mass ratio of the active pharmaceutical ingredient contained in the granule B to phenylephrine hydrochloride is in a range of 20-80:1, and a mass ratio of the active pharmaceutical ingredients contained in the granule C to phenylephrine hydrochloride is in a range of 1.2-4.8:1.

18. The phenylephrine hydrochloride-containing tablet of claim 13, further comprising adding an excipient during the preparation of the phenylephrine hydrochloride-containing tablet, and the excipient comprises calcium silicate and other common excipients.

19. The phenylephrine hydrochloride-containing tablet of claim 13, wherein the other common excipients comprise at least one selected from the group consisting of:
a stabilizer;
a filler;
a lubricant;
a defoamer;
a flavoring agent;
a pigment;
an acid source;
a sweetener;
a binder; and
an alkali source.
